(19) United States
(12) Patent Application Publication
(10) Pub. No.: US 2014/0276319 A1
(43) Pub. Date: Sep. 18, 2014

(54) ORTHOPEDIC SUPPORT SYSTEM

(71) Applicants: Tamer Ibrahim, Boonton, NJ (US); David Christian Mueller, Chatham, NJ (US)

(72) Inventors: Tamer Ibrahim, Boonton, NJ (US); David Christian Mueller, Chatham, NJ (US)

(21) Appl. No.: 13/838,983

(22) Filed: Mar. 15, 2013

(51) Int. Cl. A61F 5/01

(52) U.S. Cl. CPC A61F 5/0127
USPC 602/28

(57) ABSTRACT

The present invention relates to devices for relieving soreness, pain and/or discomfort of the foot arising from a variety of painful conditions and/or injuries. The devices of the invention provide support for the foot, are affordable, minimally visible, do not interfere with the fit of normal footwear, and are easily and accurately applied. In some embodiments, the invention relate to kits comprising the devices and an application guide for directing placement of the devices.

### FIELD OF THE INVENTION

The present invention relates to devices for relieving soreness, pain and/or discomfort of the foot, including soreness, pain and/or discomfort associated with plantar fasciitis, acute ankle sprains, acute gastrocnemius strains, Achilles tendonitis, Sever's disease, calcaneal apothysitis, foot drop, and other painful conditions or injuries of the foot. In some embodiments, the invention relate to kits comprising the devices and an application guide for placement of the devices.

More particularly, the present invention relates to an affordable device that is easily and accurately applied, provides support for the foot, and has minimal visibility.

### BACKGROUND

There are a variety of products on the market for the relief of painful conditions of the foot. These include over-the-counter arch supports, supportive insoles, and custom-fit orthotic devices. Each of these possesses disadvantages. For example, arch supports and supportive insoles vary widely in fit and comfort, and thus potentially require the purchase of several products to find one that has the best performance for a given individual. In addition, they require either the purchase of multiple pairs of supports, or the inconvenience of having to switch a single pair between multiple pairs of shoes. Custom fit orthotic devices can be effective, but are much more expensive.

Wraps can also work to provide support and cushioning to the foot. However, while many wraps are designed to support the ankle joint and the arch, most do not provide support under the foot. Moreover, since different conditions may require the different application of the wrap, both in terms of placement and size of the of the wrap, for maximum efficiency and efficacy, the wraps require application by professionals such as doctors, personal trainers or physical therapists.

While several wraps are available that allow for self-application, many require cutting of the wrap material with scissors, which introduces both inconvenience and opportunity for injury. Some wraps made from kinesiology tape do not require cutting, but these tend to be designed for specific conditions, such as acute injury or sprains, post-surgical applications. In addition, wraps, including those made of kinesiology tape, tend to be bulky, thus interfering with the fit of shoes, and also unsightly, especially for women wearing office and/or fashion footwear.

Accordingly, it would be of great benefit to have a wrap that can be easily and accurately applied by the user, and that is minimally visible and does not interfere with the fit of normal footwear. The present invention is directed to these, as well as other, important ends.

### BRIEF DESCRIPTION OF THE DRAWINGS

FIGS. 1A, 1B and 1C depict application guides according to one embodiment of the present invention.

FIGS. 2A and 2B depict an embodiment of the flexible support members-releasable backing assembly of the invention, showing flexible support members that are rectangles.

FIG. 3 depicts an embodiment of the flexible support members-releasable backing assembly of the invention, showing flexible support members that are elongated ovals.

FIG. 4 depicts an embodiment of the flexible support members-releasable backing assembly of the invention, showing flexible support members that are butterfly-shaped.

FIG. 5 depicts a foot support device in accordance with one embodiment of the present invention.

FIG. 6A depicts a medial side and sole-view (area under the dashed line) of a right foot wearing the foot support device of the present invention. FIG. 6B depicts a lateral side and sole-view (area under the dashed line) of a right foot wearing the foot support device of the present invention.

FIGS. 7A and 7B depict a combination application guide and flexible support member-releasable backing assembly having two releasable backings according to a further embodiment of the present invention.

FIGS. 7C and 7D depict a combination application guide and foot support device-releasable backing assembly having two releasable backings according to a further embodiment of the present invention.

FIGS. 8A and 8B depict a combination application guide and foot support device-releasable backing assembly having one releasable backing according to a further embodiment of the present invention.

### SUMMARY OF THE INVENTION

The present invention provides orthopedic support systems that are minimally visible and do not interfere with the fit of normal footwear. The orthopedic support systems are especially suitable for “on the fly” applications, as they are easily and accurately applied by the user.

In embodiment (I) according to the invention, kits are provided comprising an orthopedic support system, the kit comprising a plurality of identical or non-identical flexible support members disposed on a releasable backing, wherein each of the flexible support members:

a) has a first side and a second side;

b) has independently a substantially rectangular, oval or butterfly shape;

c) is comprised of an elastomeric material; and

d) has disposed upon the first side thereof an adhesive which attaches the flexible support member to the releasable backing, wherein the adhesive is substantially retained on the first side of the flexible support members upon release of the flexible support member from the backing; and

an application guide, which directs the placement of the flexible support members on the foot of a subject. In some preferred embodiments, the kits comprise three, four or five flexible support members.

In some embodiments, the application guide directs the placement of at least three flexible support members at positions on the foot selected from 1) posteriorly, from the inferior aspects of the medial to lateral malleoli; 2) plantarly, from the area overlying the medial calcaneal tubercle to the area overlying the lateral calcaneus; 3) plantarly, from the mid arch overlying the navicular tuberosity to the lateral midfoot overlying the area of the styloid process of the fifth metatarsal; 4) plantarly, from proximal to the first metatarsal head to the proximal fifth metatarsal head; and 5) approximately mid-way in between (3) and (4) above.

In some embodiments, the application guide directs the placement of at least three flexible support members in positions (1), (2) and (3) described above. In some embodiments, the application guide directs the placement of at least three flexible support members as shown for members (101), (102) and (103) in any of FIGS. 1A, 1B and 1C. In some embodiments, the application guide directs the placement of at least three flexible support members as shown for members (101), (102) and (104) in any of FIGS. 1A, 1B and 1C. In some such embodiments, the application guide further directs the placement of a fourth flexible support member, and optionally a fifth flexible support member, preferably as shown for FIGS. 1A, 1B and 1C.

In some embodiments, the application guide directs the placement of the flexible support members substantially in accordance with any of FIGS. 1A, 1B and 1C.

In some embodiments, the flexible support members have substantially the same shape, and in some embodiments, one or more of the flexible support members is of a different shape. For example, in some embodiments, each of the flexible support members can independently have substantially rectangular, butterfly or oval shape, as depicted in FIGS. 2A, 3 and 4.

In some of each of the foregoing embodiments, the flexible support members preferably have a maximum length of about 15-20 centimeters, and a maximum width of about 3-4 centimeters.

In some embodiments, each of the flexible support members is attached to the same releasable backing. In some embodiments, one or more of the flexible support members are attached to a different releasable backing. In some embodiments, the release of the flexible support members is facilitated by one or more of perforations, pull tabs, or cut outs.

In some embodiments, the releasable backing is a flexible roll.

In some embodiments, the orthopedic support system further comprises a second releasable backing, wherein:

the second releasable backing is disposed on the second side of the flexible support members;

the second releasable backing has disposed thereon a substantially life-size representation of a human foot or footprint; and

the flexible support members are attached to the releasable backing at the positions on the representation of the human foot or footprint corresponding to the intended area of placement of the flexible support members on the foot of a subject. In some embodiments, one or both of the first and second releasable backings is a firm or semi-firm card, a paper strip, a plastic strip, a paper sheet or a plastic sheet. In some embodiments, the firm or semi-firm card, or paper or plastic sheet can be substantially rectangular, triangular, square, oval, round, diamond-shaped or footprint-shaped.

In some embodiments, the flexible support members are comprised of elastic therapeutic tape. In some embodiments, the flexible support members are comprised of one or more of neoprene tape, silicone tape, latex tape, rubber tape, fabric tape or vinyl tape, with vinyl tape being preferred.

In some embodiments, the first and second releasable backings are independently selected from a firm or semi-firm card, a paper strip, a plastic strip, a paper sheet or a plastic sheet.

In some embodiments, the first and second releasable backings can independently be substantially rectangular, triangular, square, oval, round, diamond-shaped or footprint-shaped.

In accordance with a further embodiment (II), the invention provides orthopedic support systems comprising a foot support device attached to a releasable backing; wherein the foot support device:

a) is comprised of an elastomeric material;

b) has a first side and a second side;

c) has a shape that comprises a body portion and a plurality of flexible support members protruding from the body portion, wherein each of the flexible support members independently has a substantially rectangular, oval or butterfly shape; and wherein:

the first side of the foot support device has disposed thereon an adhesive which attaches the foot support device to the releasable backing; and

the adhesive is substantially retained on the first surface of the foot support device upon release of the foot support device from the releasable backing. In some embodiments, the orthopedic support system further comprises an application guide, which directs the placement of the foot support device on the foot of a subject.

In some such embodiments, the foot support device has six, eight or ten flexible support members. In some embodiments, each of the flexible support members independently has a substantially rectangular, oval or butterfly shape. In some embodiments, the foot support device is comprised of vinyl tape material.

In some embodiments, the protruding flexible support members are substantially identical in shape, and in some embodiments, one or more of the flexible support members is of a different shape, for example substantially rectangular, butterfly or oval shape.

In some embodiments, the foot support device has substantially the shape shown in any of FIG. 5, 6A, 6B, 7C or 8A.

In some embodiments, the foot support device is comprised of elastic therapeutic tape. In some embodiments, the foot support device is comprised of one or more of neoprene tape, silicone tape, latex tape, rubber tape, fabric tape or vinyl tape, with vinyl tape being preferred.

In some embodiments, the orthopedic support system further comprises a second releasable backing disposed on the second side of the foot support device, wherein:

i) the first side of the foot support device is attached to the first releasable backing by an adhesive that is substantially retained on the foot support device upon removal of the first releasable backing; and

ii) the second side of the foot support device is attached to the second releasable backing by an adhesive that is substantially retained on the second releasable backing upon removal of the support device from the second releasable backing.

In some further embodiments, the second releasable backing has disposed thereon a representation of a human footprint, and the foot support device is attached to the second releasable backing at the position on the representation of the human foot or footprint corresponding to the intended area of placement of the foot support device on the foot of a subject. In some embodiments, the representation of the human foot or footprint is substantially life-size. In some embodiments, the foot support device has substantially the shape shown in any of FIG. 5, 6A, 6B, 7C or 8A.

In some embodiments, the first and second releasable backings are independently selected from a firm or semi-firm card, a paper strip, a plastic strip, a paper sheet or a plastic sheet. In some embodiments, the firm or semi-firm card, paper strip, plastic strip, paper sheet or plastic sheet can be substantially rectangular, triangular, square, oval, round, diamond-shaped or footprint-shaped.

In some embodiments, the present invention provides methods for providing relief from foot pain and/or discomfort, comprising applying the orthopedic support systems described herein to the foot of a subject.

### DETAILED DESCRIPTION

The present invention provides orthopedic support systems that are minimally visible and do not interfere with the fit of normal footwear. The orthopedic support systems are especially suitable for “on the fly” application, because they are easily and accurately applied by the user.

As used herein, the term “substantially rectangular” is intended to mean having two approximately parallel sides, and width shorter than length. The side joining the two parallel sides can be any convenient angle relative to the two approximately parallel sides. “Substantially rectangular” also includes the structures having generally parallel curved sides, as shown for the flexible support members in FIGS. 1A, 1B, 1C, 5, 6A and 6B. As used herein, the term “substantially square” is intended to mean having two sets of approximately parallel and equal length sides. As used herein, the term “substantially triangular” is intended to mean having three approximately straight sides. Each of the foregoing terms is intended to include minor variations from the pure geometric shape, particularly in regard to corners. Thus, the terms are intended to include members having rounded corners, instead of sharp corners. Such rounded corners are advantageous in that they minimize both irritation to the skin, and the possibility of the corner being lifted up by contact with clothing and/or shoe surfaces.

As used herein, the term “substantially oval” is intended to mean having approximately ovular, for example as shown in FIG. 3. As used herein, the term “butterfly shaped” is intended to mean having an increase in width towards the extremity of the flexible support member, and preferably a rounded terminus, as shown for example in FIG. 4.

As used herein, the term “substantially retained” when used to refer to an adhesive layer, is meant to indicate that the adhesive remains on the indicated flexible support member, foot support device or release layer, except for some small unintended amount of adhesive that may remain on the other surface that is an inherent consequence of the manufacturing process.

As used herein, the term “substantially life-size” when used to refer to a representation of a human foot or footprint is intended to mean approximately the size of a human foot of a given shoe size.

As used herein, the terms “substantially in accordance” or “substantially as shown for” when used to refer to a Figure herein, is intended to mean having the same general shape and features as shown the indicated Figure.

In embodiment (I) according to the invention, kits are provided comprising an orthopedic support system, the kit comprising a plurality of identical or non-identical flexible support members disposed on a releasable backing, wherein each of the flexible support members:

a) has a first side and a second side;

b) has independently a substantially rectangular, oval or butterfly shape;

c) is comprised of an elastomeric material; and

d) has disposed upon the first side thereof an adhesive which attaches the flexible support member to the releasable backing, wherein the adhesive is substantially retained on the first side of the flexible support members upon release of the flexible support member from the backing; and an application guide, which directs the placement of the flexible support members on the foot of a subject. In some preferred embodiments, the kits comprise three, four or five flexible support members.

Generally, the flexible support members of the kits of the invention are provided attached to a releasable backing (or substrate) from which they can be released. Preferably, the flexible support members are attached to the backing with an adhesive. In some preferred embodiments, upon release from the backing, the flexible support members retain adhesive that then functions to attach the flexible support member to the foot of the subject.

FIG. 2A shows a releasable backing (7) having flexible support members (8) attached thereto. FIG. 2B shows a detail of FIG. 2A. Upon the removal of flexible support member (8) from releasable backing (7), adhesive layer (8a) is substantially retained on flexible support member (8). Then, in accordance with the invention, the application guide, for example as shown in one or more of FIGS. 1A, 1B and/or 1C, directs the placement of the flexible support members on the foot of a subject, wherein the adhesive attaches the flexible support members to the foot at the locations shown in the application guide. Preferably, the flexible support members are stretched slightly as they are applied, to impart a small degree of tension across the bottom of the foot.

FIGS. 1A, 1B and 1C show application guides according to the invention. FIG. 1A shows an application guide (1) that contains a representation of a human foot or footprint (2), and a representation of flexible support members (101)-(105) at the locations where they are intended to be placed on a foot of a subject. FIG. 1B shows an application guide (4) that contains a medial side view of a right human foot (5), including the location of the medial malleolous, (106), and a representation of flexible support members (101)-(105) at the locations where they are intended to be placed on a foot of a subject. FIG. 1C shows an application guide (4a) that contains a lateral side view of a right human foot (5) including the location of the lateral malleolous, (106a), and a representation of flexible support members (101)-(105) at the locations where they are intended to be placed on a foot of a subject. In some embodiments, the kits of the invention include one or more application guides in accordance FIGS. 1A, 1B and/or 1C. In some embodiments, the application guide comprises one or more of the footprint and side-views as shown in FIGS. 1A, 1B and/or 1C.

In some embodiments, the application guide depicts a substantially life-size representation of the human foot, with the placement of the flexible support members clearly demarked as shown for example in FIGS. 1A, 1B and 1C. In such embodiments, the intended placement of the flexible support members is facilitated by placing the foot on or next to the depiction on the application guide, and placing the flexible support members on the foot in the same location as shown on the application guide.

In some embodiments, the application guide directs the placement of at least three flexible support members at positions on the foot selected from 1) posteriorly, from the inferior aspects of the medial to lateral malleoli; 2) plantarly, from the area overlying the medial calcaneal tubercle to the area overlying the lateral calcaneus; 3) plantarly, from the mid arch overlying the navicular tuberosity to the lateral midfoot overlying the area of the styloid process of the fifth metatarsal; 4) plantarly, from proximal to the first metatarsal head to the proximal fifth metatarsal head; and 5) approximately mid-way in between (3) and (4) above. In some embodiments, the application guide directs the placement of at least three flexible support members in positions (1), (2) and (3) described above. In some embodiments, the application guide directs the placement of at least three flexible support members as shown for members (101), (102) and (103) in any of FIGS. 1A, 1B and 1C. In some embodiments, the application guide directs the placement of at least three flexible support members as shown for members (101), (102) and (104) in any of FIGS. 1A, 1B and 1C. In some embodiments, the application guide further directs the placement of a fourth flexible support member, and optionally a fifth flexible support member, preferably as shown for FIGS. 1A, 1B and 1C.

In some embodiments, the application guide directs the placement of the flexible support members substantially in accordance with any of FIGS. 1A, 1B, and 1C. It will be appreciated that some degree of overlap of the flexible support members is acceptable, and contemplated by the present invention.

In some embodiments (I) according to the invention, the orthopedic support system further comprises a second releasable backing, which is disposed on the second side of the flexible support members. In some embodiments, the second releasable backing has disposed thereon a substantially life-size representation of a human foot or footprint; and the flexible support members are attached to the releasable backing at the positions on the representation of the human foot or footprint corresponding to the intended area of placement of the flexible support members on the foot of a subject.

FIGS. 7A and 7B depict a combination application guide and flexible support member-releasable backing assembly (13) having two releasable backings according to a further embodiment of the present invention. Flexible support members with adhesive and releasable backing layers (15) are disposed over substantially life-size footprint representation (14) at the intended point of placement on the foot of the subject. FIG. 7B is a magnified view of a section (16) of a flexible support member with adhesive and releasable backing layers disposed on second releasable backing (13a). As shown in FIG. 7B, flexible support member (18) is attached to second releasable backing (13a) by adhesive layer (17), and flexible support member (18) is attached to first releasable backing (20) by adhesive layer (19).

Upon removal of first releasable backing (20), adhesive layer (19) is substantially retained on flexible support member (18). The subject then places their foot on the footprint representation (14), and lifts the ends of flexible support member (18) away from the second releasable backing (13a), and stretches flexible support member 18 as it is applied to the foot, whereupon the adhesive layer (17) is substantially retained on second releasable backing (13a). This process is repeated for each flexible support member (18), and the second releasable backing (13a) is then peeled away and discarded.

In some embodiments, each of the flexible support members (18) is attached to a separate first releasable backing (20), as depicted in FIGS. 7A and 7B. In some such embodiments, each of the first releasable backings (20) conform to the same shape as the second side of the flexible support members to which they are attached, as depicted in FIG. 7B. In some further embodiments, each of the flexible support members (18) is attached to a single first releasable backing (20), thereby facilitating the removal of the first releasable backing from all three (or more) flexible support members substantially simultaneously.

In some embodiments, each of the flexible support members of the kits of the invention are attached to the same first and/or second releasable backing. In further embodiments, one or more of the flexible support members of the kits of the invention are attached to a different first and/or second releasable backing. The first and second releasable backings can be any of a variety of materials and shapes. Nonlimiting examples of first and second releasable backings according to the invention include firm (i.e. rigid) or semi-firm (i.e., flexible) cards, strips and sheets, each of which can be any of a variety of materials, for example and not limitation, plastic, paper or cardboard. In some embodiments, the first and second releasable backings are each independently substantially rectangular, triangular, square, oval, round, diamond-shaped or footprint-shaped. In some embodiments, one or both of the first and second releasable backing is a flexible roll. In some embodiments, the release of the flexible support members from one or both of the releasable backings is facilitated by one or more of perforations, pull tabs, cut outs, etc. as are known in the art.

The flexible support members of the kits of the invention can have any of a wide variety of shapes. Suitable shapes include rectangles, preferably with rounded corners as shown in FIGS. 1A, 1B, 1C and 2A, ovals as shown in FIG. 3, and butterfly-shape as shown in FIG. 4. In some of each of the foregoing embodiments of the present invention described herein, each of the flexible support members can be substantially identical in shape, or one or more of the flexible support members can have a different shape. In some preferred embodiments, one or more of the flexible support members have substantially the shape depicted in any of FIGS. 2A, 3 and 4.

In some embodiments, the flexible support members preferably have a maximum length of about 15-20 centimeters, and a maximum width of about 3-4 centimeters.

In some embodiments, the flexible support members are comprised of elastic tape, which can be but is not necessarily therapeutic tape, for example and not limitation, one or more of neoprene tape, silicone tape, latex tape, rubber tape, fabric tape or vinyl tape, with vinyl tape being preferred.

In accordance with further embodiments (II), the invention provides orthopedic support systems comprising a foot support device attached to a releasable backing; wherein the foot support device:

a) is comprised of an elastomeric material;

b) has a first side and a second side;

c) has a shape that comprises a body portion and a plurality of flexible support members protruding from the body portion, wherein each of the flexible support members independently has a substantially rectangular, oval or butterfly shape; and wherein the first side of the foot support device has disposed thereon an adhesive which attaches said foot support device to said releasable backing; and wherein:

the adhesive is substantially retained on the first surface of the foot support device upon release of the foot support device from the releasable backing. In some embodiments, the orthopedic support system optionally comprises an application guide, which directs the placement of the foot support device on the foot of a subject. In some embodiments, the foot support device has six, eight or ten flexible support members.

In some embodiments, each of the flexible support members independently has a substantially rectangular, oval or butterfly shape.

FIG. 5 shows a foot support device for use in the orthopedic support system of the invention. FIG. 6A shows a medial side and sole-view (area under dashed line (312) of a right foot wearing the device of the present invention. Foot support device 200 has a body portion 300 and protruding flexible support members (301)-(310). In accordance with the invention, releasable backing (311) is removed by the end user, revealing an adhesive layer that remains attached to foot support device (200). Then the body portion of the foot support device is placed on the bottom of the foot, while aligning flexible support members (301) and (302) such that they proceed posteriorly, from the inferior aspects of the medial to lateral malleoli, as shown in FIGS. 6A and 6B. The remaining the flexible support members are then affixed to achieve placement of the device in accordance with FIG. 6A and/or FIG. 6B. Preferably, the flexible support members are slightly stretched as they are applied, to retain a small degree of tension after placement on the foot. It will be appreciated that the shape of the foot support device, and in particular the placement of the protruding flexible support members in relation to each other, and to the body portion of the foot support device, is selected to achieve the appropriate placement of the body portion and flexible support members of the foot of the subject. Thus, the foot support devices of the invention provide the benefits of the foregoing embodiments (I) of the invention that comprise individual flexible support members, but in a single device, which is easier and more convenient to apply.

FIGS. 8A and 8B depict a combination application guide and foot support device-releasable backing assembly (21) having one releasable backing (22) according to a further embodiment of the present invention. Foot support device (200) is attached to a releasable backing (22) via adhesive layer (8b). Upon the removal of foot support device (200) from releasable backing (22), adhesive layer (8b) is substantially retained on foot support device (200). In some embodiments, the releasable backing (22) has disposed thereon a substantially life size representation of the human foot or footprint (14) corresponding to the intended area of placement of the foot support device on the foot of a subject. Thus, in accordance with the invention, after removal from releasable backing (22), foot support device (200) is placed on the foot of the subject as described above, and in accordance with the placement shown on foot representation (14). Preferably, the flexible support members are stretched slightly as they are applied, to impart a small degree of tension across the bottom of the foot.

In some embodiments (II) according to the invention, the orthopedic support system further comprises a second releasable backing disposed on the second side of the foot support device, wherein:

i) the first side of the foot support device is attached to the first releasable backing by an adhesive that is substantially retained on the foot support device upon removal of the first releasable backing; and

ii) the second side of the foot support device is attached to the second releasable backing by an adhesive that is substantially retained on the second releasable backing upon removal of the support device from the second releasable backing.

FIGS. 7C and 7D depict a combination application guide and foot support device-releasable backing assembly (13) having a first releasable backing (20a) and a second releasable backing (22) according to a further embodiment of the present invention. As shown in detail FIG. 7B, the first side of foot support device (200) is attached to first releasable layer (20a) by adhesive layer (19a). The second side of foot support device (200) is attached to second releasable layer (22) by adhesive layer (17a). In some embodiments, second release layer 22 has disposed thereon a substantially life-size footprint representation (14) at the intended point of placement on the foot of the subject. Upon removal of first releasable backing (20a), adhesive layer (19a) is substantially retained on foot support device (200). The subject then places their foot on the footprint representation (14), and lifts the ends of the support members away from the second releasable backing (22), and stretches the flexible support members as they are applied to the foot, whereupon the adhesive layer (17a) is substantially retained by second releasable backing (22). This process is repeated for each flexible support members, and the second releasable backing (22) is then peeled away and discarded. In some embodiments, the foot support device has substantially the shape shown in any of FIG. 5, 6A, 6B, 7C or 8A.

The size and shape of the body portion of the foot support device of the invention can vary. In some embodiments, the body portion of the foot support device of the invention can cover essentially the whole width of the bottom of the foot. In other embodiments, the body portion is considerably less wide, and can be as narrow as 1 centimeter, or less. It will be appreciated that the body portion is intended to maintain the placement of the flexible support members relative to each other, and to provide a convenient and effective alternative to the individual placement of flexible support members of embodiment (I) of the invention. Thus, the flexible support member pairs (301)-(302), (303)-(304), (305)-(306), (307)-(308) and (309)-(310) of embodiment (II) of the invention are each intended to function, through the body portion, similarly to the members (101)-(105) of embodiment (I). The foot support devices of the invention are especially convenient to apply in that once the flexible support members designated (301) and (302) in FIG. 5 (or its mirror image, for a right foot) are placed, the relative positions of the remaining flexible support members are then fixed.

The size and shape of the flexible support members of the foot support devices of the invention can vary. In some embodiments, they are each substantially rectangular as shown for members (301)-(310) and in, for example, FIGS. 5, 6A, 6B and 8A. Other suitable shapes include, for example and not limitation, rectangular, oval or butterfly shapes. It will be appreciated that several parameters of the foot support devices of the invention will vary depending upon the size of the foot to which the foot support device is to be attached. These include, for example, the length of the flexible support members, and the shape and angle of the flexible support members relative to the body portion, and to the positions of the other flexible support members. In some preferred embodiments, these parameters are selected for each size foot support device such that the flexible support members reside on the foot approximately at the positions shown for members (301)-(310) in FIGS. 6A and 6B.

The first and second releasable backings for the foot support devices of the invention can be any of a variety of materials and shapes. Non-limiting examples of first and second releasable backings according to the invention include firm (i.e. rigid) or semi-firm (i.e., flexible) cards, or paper strips, plastic strips, paper sheets, plastic sheets, or cardboard cards or sheets. In some embodiments, the first and second releasable backing are independently substantially rectangular, triangular, square, oval, round, diamond-shaped or footprint-shaped. In some embodiments, one or both of the first and second releasable backing is a flexible roll. In some embodiments, one or both of the first and second release backings are coextensive in shape with the foot support device. In some embodiments, the release of the flexible support members from one or both of the releasable backings is facilitated by one or more of perforations, pull tabs, cut outs, etc. as are known in the art.

The design of the orthopedic support systems of the invention differs from foot wraps in at least the aspect that the flexible support members, or at least a substantial portion of the flexible support members, are free to move independently of one another, and thus adjust to skin movement during daily activities, exercise or any other pursuits. Thus, the present orthopedic support systems are expected to provide greater comfort and benefit.

Both the first and second releasable backings for each of the embodiments of the invention can be any of a variety of releasable backing materials, including plastic, paper, cloth, etc. It is only required that the first releasable backing (e.g., (20) in FIG. 7B, (20a) in FIG. 7D, or (7) in FIG. 2B) be removable to reveal the adhesive layer disposed on the flexible support member (or foot support device), and the second releasable backing (e.g., (13a) in FIG. 7B or (22) in FIG. 7D) be removable and retain the adhesive layer (e.g., (17) in FIG. 7B, or (17a) in FIG. 7D). In some preferred embodiments, the first releasable backing is a paper or plastic tear-away strip or sheet, and the second releasable backing is a paper, cardboard or plastic flexible sheet or card, having a representation of the human foot disposed thereon.

In some embodiments, at least three of the flexible support members are attached to the second releasable backing such that when applied to the foot of a subject, they are located at positions on the foot selected from 1) posteriorly, from the inferior aspects of the medial to lateral malleoli; 2) plantarly, from the area overlying the medial calcaneal tubercle to the area overlying the lateral calcaneus; 3) plantarly, from the mid arch overlying the navicular tuberosity to the lateral midfoot overlying the area of the styloid process of the fifth metatarsal; 4) plantarly, from proximal to the first metatarsal head to the proximal fifth metatarsal head; and 5) approximately mid-way in between (3) and (4) above. In some preferred embodiments, at least three of the flexible support members are attached to the second releasable backing such that when applied to the foot of a subject, they are located at positions on the foot (1), (2), and (3), or (1), (2) and (4), as described above.

In some embodiments, the flexible support members, or the foot support device, are attached to the second releasable backing substantially at the positions on the representation of the human footprint shown in any of FIG. 1A, 7A, 7C or 8A.

The second releasable backing, e.g., (13a) or (22), can be any convenient shape, for example substantially rectangular, triangular, square, oval, round, diamond-shaped or footprint-shaped. In some preferred embodiments, the second releasable backing (13) or (22) is substantially rectangular, or is coextensive in shape and size with the area of the human footprint representation (14) and flexible support members (15) as shown in FIG. 7A, or is coextensive in shape and size with the area of the human footprint representation (14) and foot support device 200 as shown in FIGS. 7C and/or 8A.

In each of the foregoing embodiments of the present invention described herein, the flexible support members and foot support devices can be made from a variety of materials. Preferably, the flexible support members and/or foot support devices are made from materials that are strong, flexible and elastic and thermally over the range of temperatures encountered by feet—approximately 25° F. to 110° F. Preferably, in some embodiments, the flexible support members and/or foot support devices are stretched slightly when applied, and preferably retain their tendency to return to their original dimension while applied to the foot, providing a mild pulling or tension effect to the area of application. In general, suitable materials for the flexible support members and/or foot support devices of the invention include elastic tape and elastic therapeutic tape, including kinesiology tape, and preferably those composed of one or more of neoprene, silicone, latex, rubber, fabric, and vinyl, with vinyl being particularly preferred.

Thus, in some of each of the foregoing embodiments, the flexible support members and foot support devices of the invention are comprised of elastomeric vinyl tape. In some such embodiments, the elastomeric vinyl tape preferably has a thickness of from about 4 mil (i.e., about 100 μm) to about 12 mil (i.e., about 300 μm), more preferably from about 7 mil (i.e., about 180 μm) to about 10 mil. (i.e., about 250 μm).

In some embodiments, the flexible support members of the invention are composed of, or possess stretching characteristics similar to those of electrical tape, for example Scotch super 88 (catalog 6143), and Scotch® Super 33+™ Vinyl Electrical Tape. One particularly preferred elastic tape is Adhesive Tape Products PVC Electrical Insulating Tape No. 101; E52811A; 7 Mil (0.18 mm)×¾″ (19 mm) Taiwan.

In some embodiments, the application guides, flexible support members and foot support devices of a given kit are configured for a specific range of foot sizes. Thus, in some embodiments of the present invention, the kits correspond to different ranges of foot size. In some such embodiments, the kits of the invention are for foot sizes in accordance with the following Table 1:

TABLE 1

| Size | Designation |
|---|---|
| Junior 1-2 | X-Small |
| Junior 3-5 | Small |
| Adult 6-9 | Medium |
| Adult 10-13 | Large |
| Adult 14-17 | X-Large |

In some embodiments, the present invention provides methods for providing relief from foot pain and/or discomfort, comprising applying the orthopedic support systems described herein to the foot of a subject.

The orthopedic support systems of the invention provide a number of significant benefits, compared to prior devices. Because of their ease of application, they are especially suitable for “on the fly” applications, such as on the athletic field, and do not require the participation of a trainer or other professional to accurately apply the devices.

While not wishing to be bound by any particular theory, it is believed that the present invention provides it effect, at least in part, by providing a small degree of tension across the surface of the skin beneath the flexible support members, and, for embodiments II, also the body portion of the foot support device. A further significant benefit is believed to be derived from the use of flexible support members, which impart flexibility and allow the orthopedic supports of the invention to “move” with the foot as it bends and twists in everyday activity, and especially in exercise.

An additional benefit of the present invention is derived from the elastomeric tape materials contemplated for the invention. Because the materials are relatively thin, there is less tendency for the orthopedic supports of the invention to “bunch up”, and cause irritation, particularly when used under sox, of tight-fitting shoes, such as normal men and women's casual and business attire. A further benefit is that the orthopedic supports of the invention are designed to be “low profile”—i.e., their location on the foot is mostly hidden from view, even with most women's footwear.

Although the various embodiments herein may shown for either a left foot or a right foot, it is understood that the invention in all embodiments herein includes the other foot as well, which will generally be the mirror image of the embodiment shown.

Although the invention has been described in conjunction with specific embodiments thereof, it is evident that many alternatives, modifications and variations will be apparent to those skilled in the art. Accordingly, the invention as described and hereinafter claimed is intended to embrace all such alternatives, modifications and variations that fall within the spirit and broad scope of the appended claims.

What is claimed is:

1. A kit comprising an orthopedic support system, said kit comprising:
a plurality of identical or non-identical flexible support members disposed on a releasable backing, wherein each of said flexible support members:
a) has a first side and a second side;
b) has independently a substantially rectangular, oval or butterfly shape;
c) is comprised of an elastomeric material; and
d) has disposed upon the first side thereof an adhesive which attaches said member to said releasable backing;
wherein said adhesive is substantially retained on said first side of said flexible support members upon release of said flexible support member from said backing; and
an application guide, which directs the placement of said flexible support members on the foot of a subject.

2. The kit of claim 1, wherein said application guide directs the placement of at least three of said flexible support members at positions selected from the group consisting of:
a) posteriorly, from the inferior aspects of the medial to lateral malleoli;
b) plantarly, from the area overlying the medial calcaneal tubercle to the area overlying the lateral calcaneus;
c) plantarly, from the mid arch overlying the navicular tuberosity to the lateral midfoot overlying the area of the styloid process of the fifth metatarsal
d) plantarly, from proximal to the first metatarsal head to the proximal fifth metatarsal head; and
e) approximately mid-way in between (c) and (d) above.

3. The kit of claim 2, wherein said application guide directs the placement of at least four of said flexible support members.

4. The kit of claim 2, wherein said application guide directs the placement of five of said flexible support members.

5. The kit of claim 1, comprising three flexible support members, or four flexible support members, or five flexible support members.

6. The kit of claim 1, wherein said application guide directs the placement of said flexible support members substantially in accordance with any of FIGS. 1A, 1B and 1C.

7. The kit of claim 1, where said flexible support members are substantially the same shape.

8. The kit of claim 1, wherein each of said flexible support members is attached to the same releasable backing.

9. The kit of claim 8, wherein said releasable backing is a firm or semi-firm card, a paper strip, a plastic strip, a paper sheet or a plastic sheet.

10. The kit of claim 8, wherein said releasable backing is a flexible roll.

11. The kit of claim 1, wherein the orthopedic support system further comprises a second releasable backing, wherein:
said second releasable backing is disposed on said second side of said flexible support members;
said second releasable backing has disposed thereon a substantially life-size representation of a human foot or footprint; and
said flexible support members are attached to said releasable backing at the positions on the representation of the human foot or footprint corresponding to the intended area of placement of said flexible support members on the foot of a subject.

12. The kit of claim 1, wherein said flexible support members are comprised of elastic therapeutic tape.

13. The kit of claim 1, wherein said flexible support members are comprised of one or more of neoprene tape, silicone tape, latex tape, rubber tape, fabric tape or vinyl tape.

14. The kit of claim 1, wherein said flexible support members comprise vinyl tape.

15. An orthopedic support system, comprising a foot support device attached to a releasable backing; wherein the foot support device:
a) is comprised of an elastomeric material;
b) has a first side and a second side;
c) has a shape that comprises a body portion and a plurality of flexible support members protruding from said body portion, wherein each of said flexible support members independently has a substantially rectangular, oval or butterfly shape; and
wherein said first side of said foot support device has disposed thereon an adhesive which attaches said foot support device to said releasable backing; and
wherein said adhesive is substantially retained on said first surface of said foot support device upon release of said foot support device from said releasable backing, and
d) optionally comprises an application guide, which directs the placement of said foot support device on the foot of a subject.

16. The orthopedic support system of claim 15, wherein said foot support device has six flexible support members.

17. The orthopedic support system of claim 15, wherein said foot support device has eight flexible support members.

18. The orthopedic support system of claim 15, wherein said foot support device has ten flexible support members.

19. The orthopedic support system of claim 15, wherein said foot support device has substantially the shape shown in any of FIG. 5, 6A, 6B, 7C or 8A.

20. The orthopedic support system of claim 15, wherein said flexible support members have substantially rectangular shape.

21. The orthopedic support system of claim 15, wherein said flexible support members have substantially butterfly shape.

22. The orthopedic support system of claim 15, wherein said flexible support members have substantially oval shape.

23. The orthopedic support system of claim 15, wherein said foot support device is comprised of one or more of neoprene tape, silicone tape, latex tape, rubber tape, fabric tape or vinyl tape.

24. The orthopedic support system of claim 15, wherein said foot support device is comprised of vinyl tape.

25. The orthopedic support system of claim 15, further comprising a second releasable backing disposed on said second side of said foot support device; wherein:
i) said first side of said foot support device is attached to said first releasable backing by an adhesive that is substantially retained on said foot support device upon removal of said first releasable backing; and
ii) said second side of said foot support device is attached to said second releasable backing by an adhesive that is substantially retained on said second releasable backing upon removal of said support device from said second releasable backing.

26. The orthopedic support system of claim 25, wherein said second releasable backing has disposed thereon a representation of a human footprint, and said foot support device is attached to said second releasable backing at the position on said representation of said human footprint corresponding to the intended area of placement of said foot support device on the foot of a subject.

27. The orthopedic support system of claim 26, wherein said representation of said human footprint is substantially life-size.

28. The orthopedic support system of claim 25, wherein said foot support device is comprised of one or more of neoprene tape, silicone tape, latex tape, rubber tape, fabric tape or vinyl tape.

29. The orthopedic support system of claim 25, wherein said foot support device is comprised of vinyl tape.

30. The orthopedic support system of claim 25, wherein said foot support device has substantially the shape shown in any of FIG. 5, 6A, 6B, 7C or 8A.

31. The orthopedic support system of claim 25, wherein said first releasable backing and said second releasable backing are independently selected from a firm or semi-firm card, a paper strip, a plastic strip, a paper sheet or a plastic sheet.

32. A method for providing relief from foot pain and/or discomfort, comprising applying the orthopedic support system of claim 1 to a subject.

33. A method for providing relief from foot pain and/or discomfort, comprising applying the orthopedic support system of claim 15 to a subject.

34. A method for providing relief from foot pain and/or discomfort, comprising applying the orthopedic support system of claim 25 to a subject.
